(19) United States
(12) Patent Application Publication
(10) Pub. No.: US 2026/0184732 A1
(43) Pub. Date: Jul. 2, 2026

(54) PROCESS AND INTERMEDIATES FOR PREPARING ((1R,2S,4R)-4-((5-(4-((R)-7-CHLORO-1,2,3,4-TETRAHYDROISOQUINOLIN-1-YL)-5-METHYLTHIOPHENE-2-CARBONYL)PYRIMIDIN-4-YL)AMINO)-2-HYDROXYCYCLOPENTYL)METHYL SULFAMATE

(71) Applicant: TAKEDA PHARMACEUTICAL COMPANY LIMITED, Osaka (JP)

(72) Inventors: John Daniel BAILEY, Cambridge, MA (US); Joshua David WAETZIG, Cambridge, MA (US); Jonathan Vinh-Phu TRUONG, Cambridge, MA (US)

(21) Appl. No.: 19/131,056

(22) PCT Filed: Nov. 21, 2023
(86) PCT No.: PCT/IB2023/061737

§ 371 Date: May 19, 2025

Related U.S. Application Data
(60) Provisional application No. 63/384,657, filed on Nov. 22, 2022.
(60) Provisional application No. 63/509,952, filed on Jun. 23, 2023.

(51) Int. Cl. C07F 9/6558; C07D 409/14

(52) U.S. Cl. CPC C07F 9/65586; C07D 409/14; C07B 2200/13

(57) ABSTRACT

The present disclosure relates to a process for preparing ((1R,2S,4R)-4-((5-(4-((R)-7-chloro-1,2,3,4-tetrahydroiso-quinolin-1-yl)-5-methylthiophene-2-carbonyl)pyrimidin-4-yl)amino)-2-hydroxycyclopentyl)methyl sulfamate and to novel intermediates used in the process.

### FIELD

The present disclosure relates to a process and intermediates for preparing ((1R,2S,4R)-4-((5-(4-((R)-7-chloro-1,2,3,4-tetrahydroisoquinolin-1-yl)-5-methylthiophene-2-carbonyl)pyrimidin-4-yl)amino)-2-hydroxycyclopentyl)methyl sulfamate, which is a mechanism-based inhibitor of SUMO-activating enzyme (SAE).

### BACKGROUND

Substituted pyrimidines, such as ((1R,2S,4R)-4-((5-(4-((R)-7-chloro-1,2,3,4-tetrahydroisoquinolin-1-yl)-5-methylthiophene-2-carbonyl)pyrimidin-4-yl)amino)-2-hydroxycyclopentyl)methyl sulfamate, are inhibitors of SUMO-activating enzyme (SAE). The isolation and commercial-scale preparation of substituted pyrimidines, such as ((1R,2S,4R)-4-((5-(4-((R)-7-chloro-1,2,3,4-tetrahydroisoquinolin-1-yl)-5-methylthiophene-2-carbonyl)pyrimidin-4-yl)amino)-2-hydroxycyclopentyl)methyl sulfamate, present a number of challenges. For example, an existing method requires costly purification steps, such as column chromatography, and results in intermediates having difficult handling properties.

The compound ((1R,2S,4R)-4-((5-(4-((R)-7-chloro-1,2,3,4-tetrahydroisoquinolin-1-yl)-5-methylthiophene-2-carbonyl)pyrimidin-4-yl)amino)-2-hydroxycyclopentyl)methyl sulfamate, solid state forms thereof, and the use of this compound as an inhibitor of SAE, and in the treatment of proliferative, inflammatory, cardiovascular, and neurodegenerative diseases or disorders are diclosed in International Publication No. WO 2016/004136. Also disclosed in this published PCT is a method for synthesizing ((1R,2S,4R)-4-((5-(4-((R)-7-chloro-1,2,3,4-tetrahydroisoquinolin-1-yl)-5-methylthiophene-2-carbonyl)pyrimidin-4-yl)amino)-2-hydroxycyclopentyl)methyl sulfamate.

The known method for preparing ((1R,2S,4R)-4-((5-(4-((R)-7-chloro-1,2,3,4-tetrahydroisoquinolin-1-yl)-5-methylthiophene-2-carbonyl)pyrimidin-4-yl)amino)-2-hydroxycyclopentyl)methyl sulfamate, required costly purification steps, such as column chromatography, and provided intermediates with difficult handling properties, in particular, attempted isolation of many of the process intermediates led to the formation of amorphous foams. It is difficult to remove solvent from the process intermediate foams, leading to difficulty in accurately weighing the intermediate and charging reaction vessels with appropriate amount of reactants for the following steps in the process.

Accordingly, there is a need for a more efficient method for preparing ((1R,2S,4R)-4-((5-(4-((R)-7-chloro-1,2,3,4-tetrahydroisoquinolin-1-yl)-5-methylthiophene-2-carbonyl)pyrimidin-4-yl)amino)-2-hydroxycyclopentyl)methyl sulfamate that avoids costly and undesirable challenges.

### BRIEF SUMMARY

Disclosed herein is an improved process for preparing ((1R,2S,4R)-4-((5-(4-((R)-7-chloro-1,2,3,4-tetrahydroisoquinolin-1-yl)-5-methylthiophene-2-carbonyl)pyrimidin-4-yl)amino)-2-hydroxycyclopentyl)methyl sulfamate, as well as novel intermediates useful for its synthesis. The method and novel intermediates enable efficient manufacturing of ((1R,2S,4R)-4-((5-(4-((R)-7-chloro-1,2,3,4-tetrahydroisoquinolin-1-yl)-5-methylthiophene-2-carbonyl)pyrimidin-4-yl)amino)-2-hydroxycyclopentyl)methyl sulfamate on a kilogram scale.

The novel reaction pathways for synthesis of ((1R,2S,4R)-4-((5-(4-((R)-7-chloro-1,2,3,4-tetrahydroisoquinolin-1-yl)-5-methylthiophene-2-carbonyl)pyrimidin-4-yl)amino)-2-hydroxycyclopentyl)methyl sulfamate employ intermediates and reaction steps that significantly improve the efficiency of the synthesis. In addition, the novel intermediate compounds (also referred to herein as Compounds of the Disclosure) allow for a more efficient process for preparing ((1R,2S,4R)-4-((5-(4-((R)-7-chloro-1,2,3,4-tetrahydroisoquinolin-1-yl)-5-methylthiophene-2-carbonyl)pyrimidin-4-yl)amino)-2-hydroxycyclopentyl)methyl sulfamate.

A previously known method for preparing ((1R,2S,4R)-4-((5-(4-((R)-7-chloro-1,2,3,4-tetrahydroisoquinolin-1-yl)-5-methylthiophene-2-carbonyl)pyrimidin-4-yl)amino)-2-hydroxycyclopentyl)methyl sulfamate that used tert-butyloxycarbonyl (Boc) resulted in intermediates that were difficult and problematic to isolate. A major issues was that many of the intermediates could not be isolated as solids and were isolated as solid foams/glasses with difficult handling properties. All of the intermediates tended to retain solvents making it difficult to obtain accurate weights. As a result, determining consistent and accurate reagent equivalents was difficult. Not being able to accurately calculate the correct amounts of reagents can be very problematic and dangerous when performing large scale production of ((1R,2S,4R)-4-((5-(4-((R)-7-chloro-1,2,3,4-tetrahydroisoquinolin-1-yl)-5-methylthiophene-2-carbonyl)pyrimidin-4-yl)amino)-2-hydroxycyclopentyl)methyl sulfamate. In addition, very labor intensive and costly column chromatography is necessary in the final step to achieve high enough purity to enable effective crystallization of ((1R,2S,4R)-4-((5-(4-((R)-7-chloro-1,2,3,4-tetrahydroisoquinolin-1-yl)-5-methylthiophene-2-carbonyl)pyrimidin-4-yl)amino)-2-hydroxycyclopentyl)methyl sulfamate. The percent yield of the crystallization is highly dependent on the purity of the ((1R,2S,4R)-4-((5-(4-((R)-7-chloro-1,2,3,4-tetrahydroisoquinolin-1-yl)-5-methylthiophene-2-carbonyl)pyrimidin-4-yl)amino)-2-hydroxycyclopentyl)methyl sulfamate being crystallized and material with lower purity can lead to crystallization failure.

In one aspect, the present disclosure relates to a process for preparing a compound of Formula III:

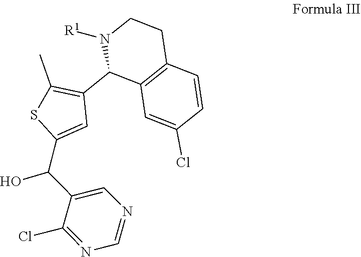

comprising reacting a compound of Formula I:

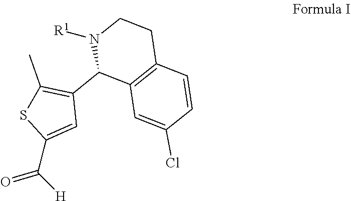

wherein R¹ is selected from diphenylphosphine oxide (DPP), dibenzosuberyl (DBS), trifluoroacetate (TFA) or —C(O)OtBu (tBoc);
with a compound of Formula II

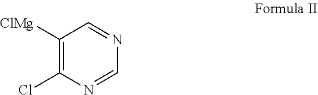

in a solvent while maintaining a reaction temperature of about −20° C. to about 10° C. to form a compound of Formula III:

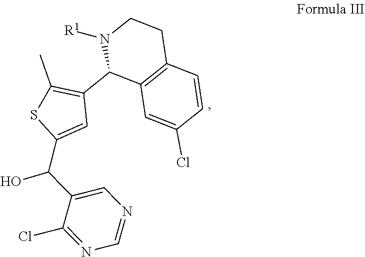

wherein R¹ is as defined above; and wherein a compound of Formula II is prepared by reacting 4-chloro-5-iodopyrimidine with isopropyl magnesium chloride-lithium chloride complex (i-PrMgCl·LiCl) in an anhydrous solvent while maintaining a reaction temperature of about −20° C. to about 10° C., for example about 0° C. to about 5° C.; adding an acidic aqueous solution to quench the reaction; and isolating a compound of Formula III.

In some embodiments, R¹ is DPP.

In another aspect, the solvent employed in the process for preparing a compound of Formula III comprises one or more polar aprotic organic solvents selected from tetrahydrofuran (THF), 2-methyl-tetrahydrofuran (2-MeTHF), cyclopentyl methyl ether (CPME), and diethyl ether.

In some embodiments, the polar aprotic solvent is THF.

In another aspect, the process for preparing a compound of Formula III comprises combining 4-chloro-5-iodopyrimidine in THE with i-PrMgCl·LiCl in THF; and the mixture of 4-chloro-5-iodopyrimidine and i-PrMgCl·LiCl in THE is thereafter combined with a compound of Formula I in THE to form a compound of Formula III.

In some embodiments, the process for preparing a compound of Formula III is quenched using an aqueous acid solution. In one aspect, the aqueous acid solution is a 10% ammonium chloride solution.

In some embodiments, the temperature for process for preparing a compound of Formula III is maintained from about 0° C. to about 5° C.

In some embodiments, a compound of Formula III is isolated by crystallization.

In some aspects, the compound of Formula III is isolated from solution by crystallization. In some aspects, the crystallization step is performed using ethanol and water.

In another aspect, the present disclosure relates to a process for preparing a compound of Formula VIII:

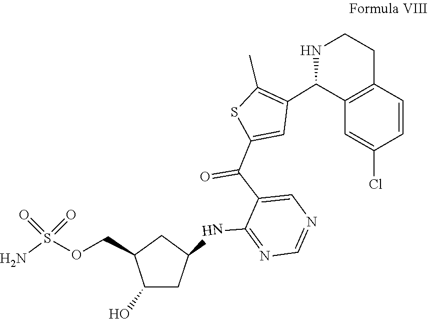 [SMILES: Cc1sc(C(=O)c2cncnc2N[C@@H]2C[C@H](COS(N)(=O)=O)[C@@H](O)C2)cc1[C@@H]1NCCc2ccc(Cl)cc21]

the process comprising oxidizing a compound of Formula III in one or more solvents to form a compound of Formula IV:

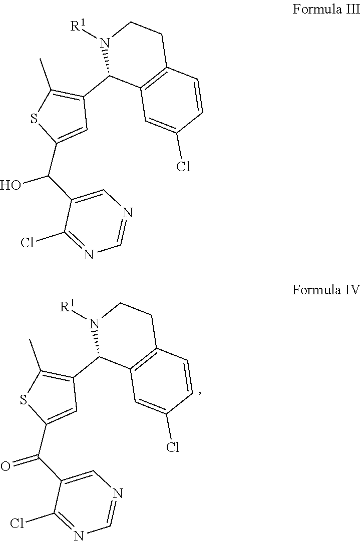

reacting a compound of Formula IV with a compound of Formula V in one or more solvents to form a compound of Formula VI:

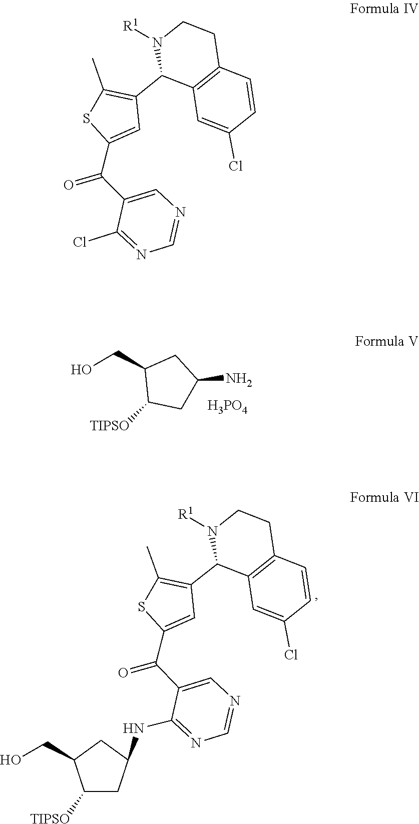

reacting a compound of Formula VI with sulfamoyl chloride in one or more solvents to form a compound of Formula VII:

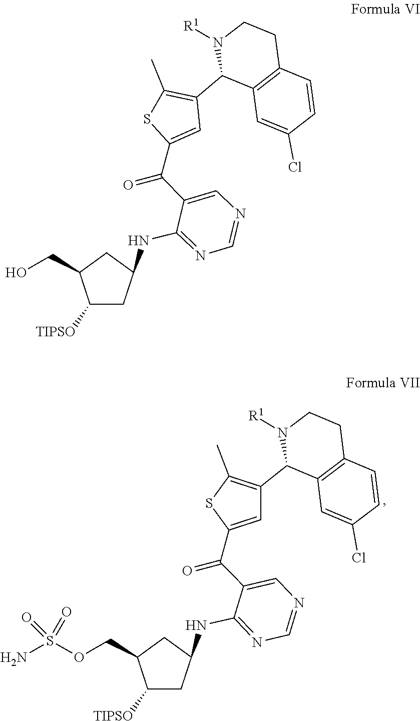

treating a compound of Formula VII with phosphoric acid in one or more solvents to form a compound of Formula VIII:

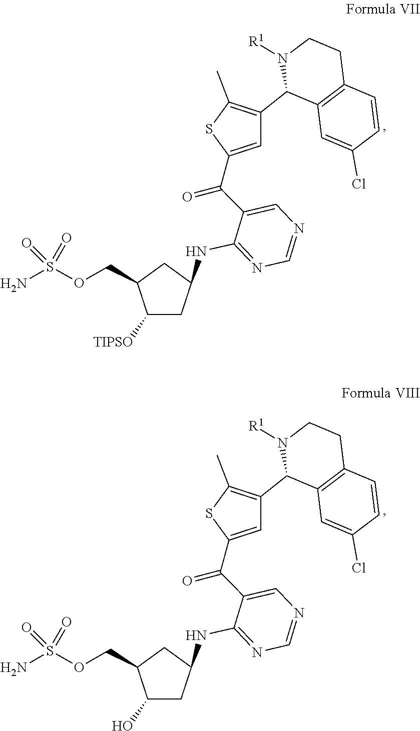

wherein for each of Formulae III, IV, V, VI and VII R¹ is DPP, DBS, TFA or —C(O)OtBu.

In some aspects, a compound of Formula VIII is re-crystallized.

In another aspect, the compound of Formula III is oxidized using hypochlorite and a catalyst selected from 9-Azabicyclo[3.3.1]nonane N-Oxyl (ABNO), 2-Azaadamantane N-Oxyl (AZADO), 4-hydroxy-2,2,6,6-tetramethylpiperidin-1-oxyl (hydroxyl-TEMPO) and 2,2,6,6-tetramethylpiperidinyloxy (TEMPO).

In another aspect, the compound of Formula III is oxidized in a solvent selected from THF, 2-MeTHF, dichloromethane (DCM), toluene, methyl ethyl ketone (MEK), methyl iso-butyl ketone (MIBK), acetone, 1-methyl-2-pyrrolidinone (NMP), N,N-dimethylformamide (DMF), and combinations thereof.

In another aspect, the compound of Formula IV is reacted with the compound of Formula V in a solvent selected from THF, 2-MeTHF, DCM, methanol (MeOH) and combinations thereof.

In another aspect, the compound of Formula VI is reacted with sulfamoyl chloride in a solvent selected from N-methyl-2-pyrrolidone, pyridine, 2-MeTHF, THF, DMF, N,N-dimethylacetamide (DMAc), water and combinations thereof.

In another aspect, a compound of Formula VII is treated with phosphoric acid in a solvent selected from acetonitrile, isopropanol, ethanol, methanol, acetone, water and combinations thereof.

In another aspect, the process for preparing a compound of Formula III is conducted in a continuous flow reactor.

In another aspect, the process for preparing a compound of Formula VIII; wherein the reaction steps for preparing the compound of Formula III are conducted in a continuous flow reactor.

In another aspect, a compound of the disclosure is a compound having Formula IX:

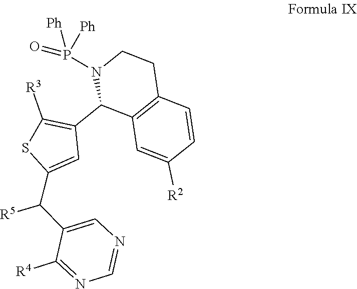

wherein
   R² is selected from H and halogen;
   R³ is selected from H and alkyl;
   R⁴ is selected from H, halogen, and —NR⁶R⁷;
   R⁵ is selected from —OH and =O;
   R⁶ and R⁷ are independently selected from H, Formula X, and Formula XI:

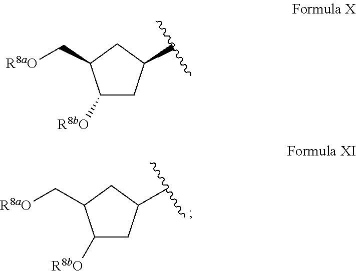

R^{8a} and R^{8b} are independently selected from H, triisopropylsilyl, trimethylsilyl, triethylsilyl, tert-butyldimethylsilyl, tert-butyldiphenylsilyl, and —S(O)₂NR⁹R¹⁰; and
R⁹ and R¹⁰ are independently selected from H and alkyl.

In another aspect, a compound of the disclosure is a compound having Formula IX; wherein
R² is selected from H and halogen;
R³ is selected from H and alkyl;
R⁴ is Cl; and
R⁵ is ═O.

In another aspect, a compound of the disclosure is a compound having Formula IX; wherein
R² is selected from H and halogen;
R³ is selected from H and alkyl;
R⁴ is —NR⁶R⁷;
R⁵ is ═O;
R⁶ and R⁷ are independently selected from H, Formula X, and Formula XI:

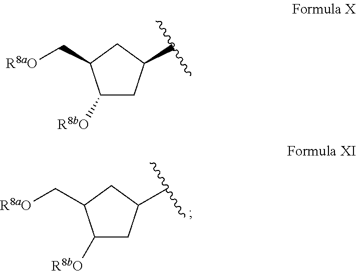

R⁸ and R^{8b} are independently selected from H, triisopropylsilyl, trimethylsilyl, triethylsilyl, tert-butyldimethylsilyl, tert-butyldiphenylsilyl, and —S(O)₂NR⁹R¹⁰; and R⁹ and R¹⁰ are independently selected from H and alkyl.

In some embodiments, a compound of the disclosure is a compound having Formula IX; wherein
R² is selected from H and halogen;
R³ is selected from H and alkyl;
R⁴ is —NR⁶R⁷;
R⁵ is ═O;
R⁶ is H;
R⁷ is Formula X:

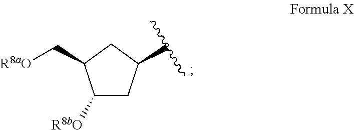

R^{8a} is —S(O)₂NR⁹R¹⁰;
R^{8b} is TIPS; and
R⁹ and R¹⁰ are H.

In some embodiments, a compound of the disclosure is a compound having Formula XII:

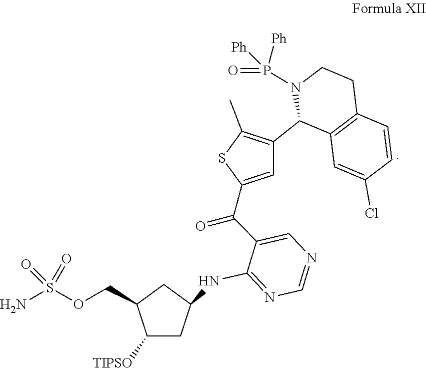

In some embodiments, a compound of the disclosure is a compound having Formula XIII:

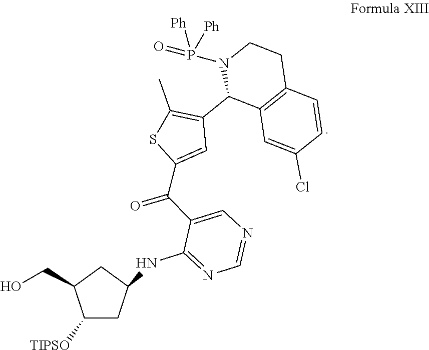

In some embodiments, a compound of the disclosure is a compound having Formula XIV:

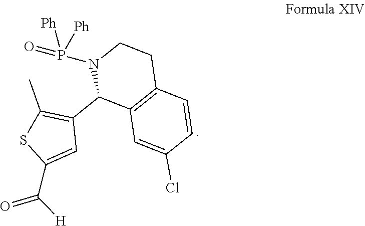 [SMILES: Cc1sc(C=O)cc1[C@H]1c2cc(Cl)ccc2CCN1P(=O)(c1ccccc1)c1ccccc1]

### DETAILED DESCRIPTION

The present disclosure generally relates to a method and intermediates for synthesizing ((1R,2S,4R)-4-((5-(4-((R)-7-chloro-1,2,3,4-tetrahydroisoquinolin-1-yl)-5-methylthiophene-2-carbonyl)pyrimidin-4-yl)amino)-2-hydroxycyclopentyl)methyl sulfamate (the compound of Formula VIII). An embodiment of the method is shown in Scheme 1:

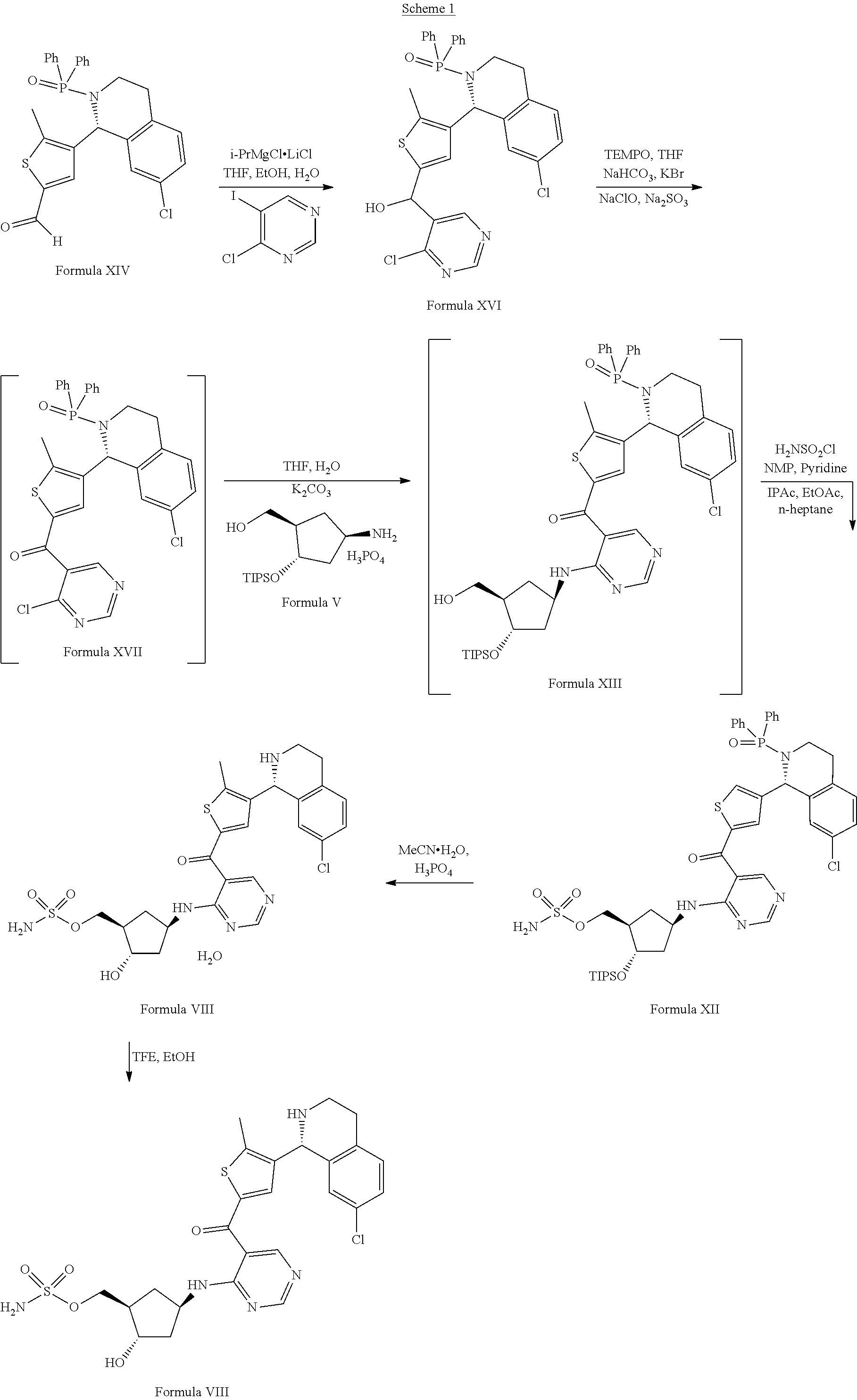

The use of the DPP protecting group, as outlined in Scheme 1, resulted in the isolation of intermediate of Formula XVI as a solid and the elimination of a column chromatography. Additionally, the use of DPP allowed for the next two intermediates of Formula XVII and Formula XIII to be isolated through extraction and used as a solution in the next steps. Finally, a large scale column chromatography was eliminated in favor of a simpler silica plug follow by crystallization which resulted in higher purity ((1R,2S,4R)-4-((5-(4-((R)-7-chloro-1,2,3,4-tetrahydroisoquinolin-1-yl)-5-methylthiophene-2-carbonyl)pyrimidin-4-yl)amino)-2-hydroxycyclopentyl)methyl sulfamate that could be used to prepare highly pure ((1R,2S,4R)-4-((5-(4-((R)-7-chloro-1,2,3,4-tetrahydroisoquinolin-1-yl)-5-methylthiophene-2-carbonyl)pyrimidin-4-yl)amino)-2-hydroxycyclopentyl)methyl sulfamate through recystallization.

In a first step of the process, a compound of Formula III:

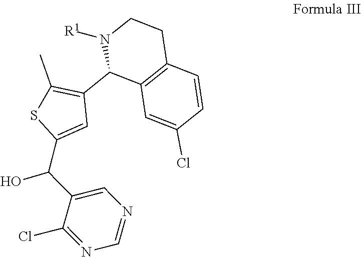

is prepared by reacting a compound of Formula I:

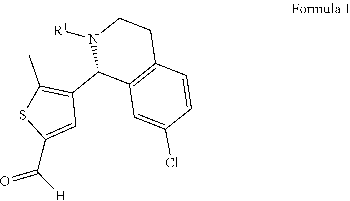

wherein R¹ is selected from diphenylphosphine oxide (DPP), dibenzyl suberyl (DBS), trifluoroacetate (TFA) or —C(O)OtBu (R¹, for each of Formulae III, IV, V, VI and VII described herein, can be DPP, DBS, TFA or —C(O)OtBu);
with a compound of Formula II

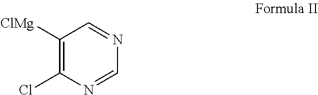

in a solvent while maintaining a reaction temperature of about −20° C. to about 10° C. to form a compound of Formula III:

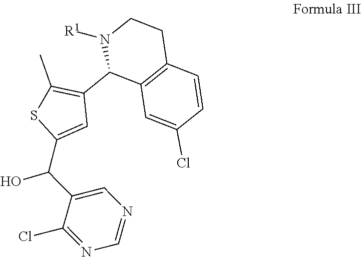

wherein R¹ is a protecting group and is defined above. The compound of Formula II is a Grignard reagent that is prepared by reacting 4-chloro-5-iodopyrimidine with isopropyl magnesium chloride-lithium chloride complex (i-PrMgCl·LiCl) in an anhydrous solvent while maintaining a reaction temperature of about −20° C. to about 10° C., or from 0° C. to about 5° C.; adding an acidic aqueous solution to quench the reaction; and isolating a compound of Formula III.

In some embodiments, R¹ is DPP. In some embodiments, R¹ is DBS. In some embodiments, R¹ is trifluoroacetate.

Suitably, the compound of Formula I is reacted with the compound of Formula II while maintaining a low reaction temperature such as about −10° C. to about 10° C., about 0° C. to about 10° C., or about 0° C. to about 5° C.

The reaction of a compound of Formula I and compound of Formula II is conducted in one or more polar aprotic organic solvents selected from tetrahydrofuran (THF), 2-methyl-tetrahydrofuran (2-MeTHF), cyclopentyl methyl ether (CPME), and diethyl ether.

In some embodiments, the polar aprotic solvent is THF. In some embodiments, the polar aprotic solvent is 2-MeTHF. In some embodiments, the polar aprotic solvent is CPME. In some embodiments, the polar aprotic solvent is diethyl ether.

In one aspect, 4-chloro-5-iodopyrimidine is combined with i-PrMgCl·LiCl in THF; the mixture of 4-chloro-5-iodopyrimidine and i-PrMgCl·LiCl in THF is thereafter combined with a compound of Formula I in THF to form a compound of Formula III.

In some embodiments, the process for preparing a compound of Formula III is conducted in a continuous flow reactor.

In another aspect, the continuous flow stream for the process for preparing a compound of Formula III contains less than or equal to 3.0% AUC of compound of Formula I.

In another aspect, the continuous flow stream for the process for preparing a compound of Formula III contains about 0.05 to about 5.0% AUC of compound of Formula II.

In another aspect, the continuous flow stream for the process for preparing a compound of Formula III contains less than or equal to 1.0% AUC of compound of Formula XV:

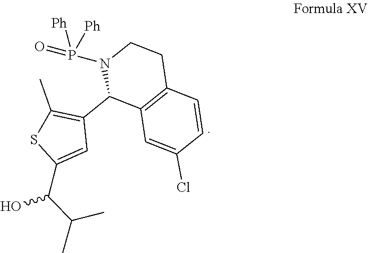 [SMILES: Cc1sc(C(O)C(C)C)cc1[C@H]1c2cc(Cl)ccc2CCN1P(=O)(c1ccccc1)c1ccccc1]

In some embodiments, the process for preparing a compound of Formula III is quenched using an aqueous acid solution. In some embodiments, the aqueous acid solution is an ammonium chloride solution. In some embodiments, the process for preparing a compound of Formula III is quenched using an aqueous acid solution of 10% ammonium chloride solution.

In some embodiments, a compound of Formula III is isolated by crystallization. In some embodiments, a compound of Formula III is crystallized from ethanol and water. In some embodiments, a compound of Formula III is crystallized from ethanol and water in a ratio of about 5:1 to 1:5 ethanol to water (% v/v). In some embodiments, a compound of Formula III is crystallized from ethanol and water in a ratio of about 2:1 to 1:2 ethanol to water (% v/v). In one embodiment, the crystallization is conducted in a solvent that is about a 1:1 ratio of ethanol to water (% v/v).

Once a compound of Formula III is isolated by crystallization, the compound of Formula III is subjected to a series of reactions to prepare a compound of Formula VIII:

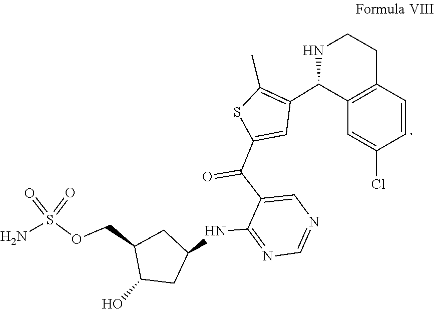 [SMILES: Cc1sc(C(=O)c2cncnc2N[C@@H]2C[C@H](COS(N)(=O)=O)[C@@H](O)C2)cc1[C@@H]1NCCc2ccc(Cl)cc21]

First, the compound of Formula III is dissolved in one or more solvents and oxidized by adding an oxidant and catalyst to form a compound of Formula IV:

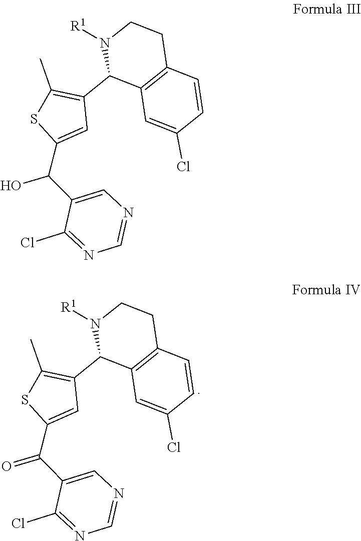

In an embodiment, the compound of Formula III is oxidized using hypochlorite and a catalyst selected from 9-Azabicyclo[3.3.1]nonane N-Oxyl (ABNO), 2-Azaadamantane N-Oxyl (AZADO), 4-hydroxy-2,2,6,6-tetramethylpiperidin-1-oxyl (hydroxyl-TEMPO) and 2,2,6,6-tetramethylpiperidinyloxy (TEMPO) to form a compound of Formula IV. In some embodiments, a compound of Formula III is oxidized using hypochlorite and the catalyst ABNO. In some embodiments, a compound of Formula III is oxidized using hypochlorite and the catalyst AZADO. In some embodiments, a compound of Formula III is oxidized using hypochlorite and the catalyst hydroxyl-TEMPO. In some embodiments, a compound of Formula III is oxidized using hypochlorite and the catalyst TEMPO.

The compound of Formula III is typcially oxidized in a solvent selected from THF, 2-MeTHF, dichloromethane (DCM), toluene, methyl ethyl ketone (MEK), methyl iso-butyl ketone (MIBK), acetone, 1-methyl-2-pyrrolidinone (NMP), N,N-dimethylformamide (DMF), and combinations thereof. In some embodiments, a compound of Formula III is oxidized in the solvent THF. In some embodiments, a compound of Formula III is oxidized in the solvent 2-MeTHF. In some embodiments, a compound of Formula III is oxidized in the solvent DCM. In some embodiments, a compound of Formula III is oxidized in the solvent toluene. In some embodiments, a compound of Formula III is oxidized in the solvent MEK. In some embodiments, a compound of Formula III is oxidized in the solvent MIBK. In some embodiments, the compound of Formula III is oxidized in the solvent acetone. In some embodiments, a compound of Formula III is oxidized in the solvent NMP. In some embodiments, the compound of Formula III is oxidized in the solvent DMF.

Suitable reaction temperatures for when the compound of Formula III is oxidized is a reaction temperature of about 0° C. to about 10° C. In some embodiments, a compound of Formula III is oxidized at a reaction temperature of about 5° C.

Thereafter, the compound of Formula IV is reacted with a compound of Formula V in one or more solvents to form a compound of Formula VI:

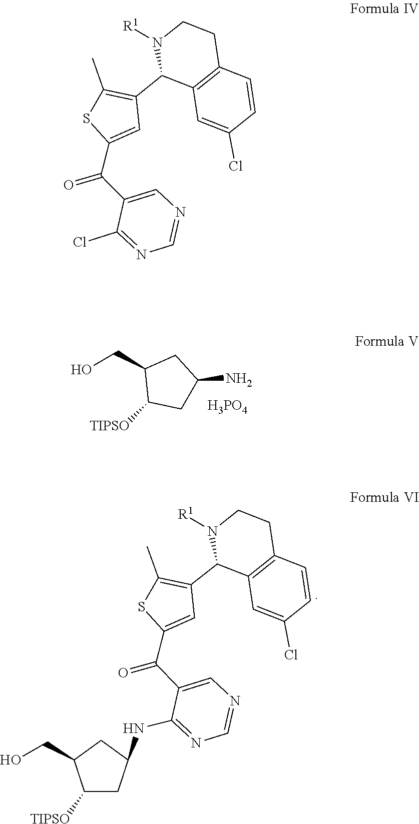

The reaction between a compound of Formula IV and a compound of Formula V is typically conducted in a solvent selected from THF, 2-MeTHF, DCM, methanol (MeOH), water or combinations thereof to form a compound of Formula VI. In some embodiments, a compound of Formula IV is reacted with a compound of Formula V in the solvent THF. In some embodiments, a compound of Formula IV is reacted with a compound of Formula V in the solvent 2-MeTHF. In some embodiments, the compound of Formula IV is reacted with a compound of Formula V in the solvent DCM. In some embodiments, a compound of Formula IV is reacted with a compound of Formula V in the solvent MeOH. In some embodiments, a compound of Formula IV is reacted with a compound of Formula V in a biphasic system of the solvent THF and water. The biphasic system allows for dissolution of the bases described below, and also aids the reaction by partitioning HCl byproduct into the aqueous phase.

In some embodiments, the compound of Formula V is the free base:

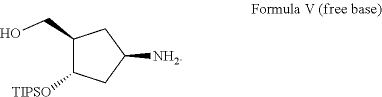

Typically, the reaction between a compound of Formula IV and a compound of Formula V in conducted in the presence of a base. In another aspect, a compound of Formula IV is reacted with a compound of Formula V in the presence of a base selected from potassium carbonate, sodium carbonate, cesium carbonate or sodium hydroxide. In some embodiments, a compound of Formula IV is reacted with a compound of Formula V in the presence of potassium carbonate. In some embodiments, a compound of Formula IV is reacted with a compound of Formula V in the presence of sodium carbonate.

In some embodiments, a compound of Formula IV is reacted with a compound of Formula V at a reaction temperature of about 25° C. to about 55° C. In some embodiments, a compound of Formula IV is reacted with a compound of Formula V at a reaction temperature of about 35° C. to about 50° C. In some embodiments, a compound of Formula IV is reacted with a compound of Formula V at a reaction temperature of about 40° C. to about 45° C. In some embodiments, a compound of Formula IV is reacted with a compound of Formula V at a reaction temperature of about 45° C.

Thereafter, the compound of Formula VI is reacted with sulfamoyl chloride in one or more solvents to sulfamoylate the compound of Formula VI and form a compound of Formula VII:

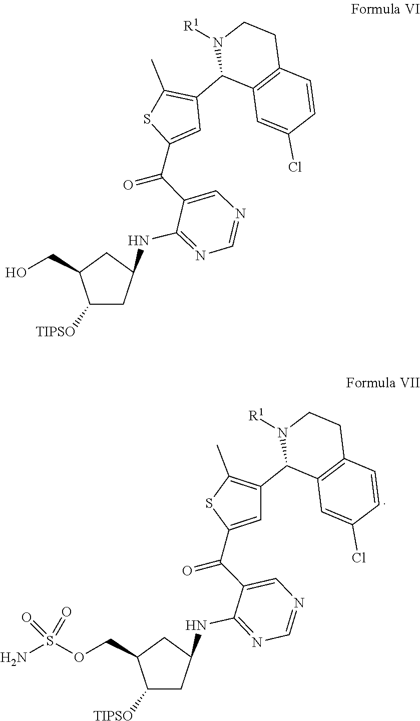

Suitably, a compound of Formula VI is reacted with sulfamoyl chloride in a solvent selected from N-methyl-2-pyrrolidone, pyridine, 2-MeTHF, THF, DMF, N,N-dimethylacetamide (DMAc), water and combinations thereof to form a compound of Formula VII. In some embodiments, a compound of Formula VI is reacted with sulfamoyl chloride in N-methyl-2-pyrrolidone. In some embodiments, a compound of Formula VI is reacted with sulfamoyl chloride in pyridine. In some embodiments, tahe compound of Formula VI is reacted with sulfamoyl chloride in 2-MeTHF. In some embodiments, a compound of Formula VI is reacted with sulfamoyl chloride in THF. In some embodiments, a compound of Formula VI is reacted with sulfamoyl chloride in DMF. In some embodiments, a compound of Formula VI is reacted with sulfamoyl chloride in DMAc.

The reaction of the compound of Formula VI and sulfamoyl chloride is typically carried out in the presence of a base. In some embodiments, a compound of Formula VI is reacted with sulfamoyl chloride in the presence of pyridine, 2,6-lutidine or collidine. In some embodiments, a compound of Formula VI is reacted with sulfamoyl chloride in the presence of pyridine. In some embodiments, a compound of Formula VI is reacted with sulfamoyl chloride in the presence of 2,6-lutidine. In some embodiments, a compound of Formula VI is reacted with sulfamoyl chloride in the presence of collidine.

The reaction of Formula VI and sulfamoyl chloride is carried out at a relative low temperature. In some embodiments, a compound of Formula VI is reacted with sulfamoyl chloride at a reaction temperature of about 0° C.

Next, the compound of Formula VII is treated with phosphoric acid in one or more solvents to deprotect the protected hydroxy and protected ring amino group of Formula VII, thereby forming a compound of Formula VIII:

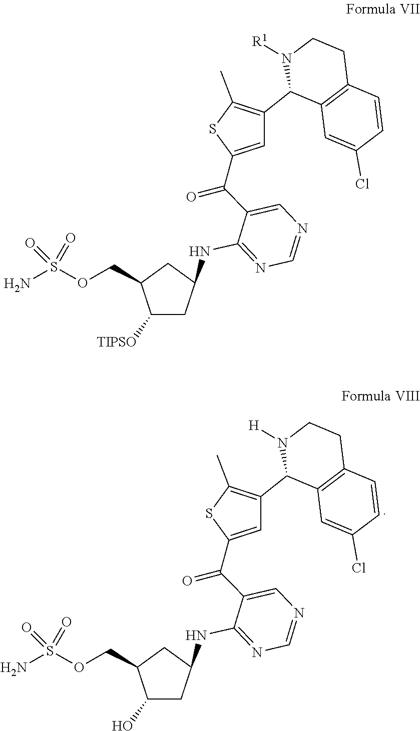

In another aspect, a compound of Formula VII is treated with phosphoric acid in a solvent selected from acetonitrile, isopropanol, ethanol, methanol, acetone, water or combinations thereof to form the compound of Formula VIII. In some embodiments, a compound of Formula VII is treated with phosphoric acid in acetonitrile. In some embodiments, a compound of Formula VII is treated with phosphoric acid in isopropanol. In some embodiments, a compound of Formula VII is treated with phosphoric acid in ethanol. In some embodiments, a compound of Formula VII is treated with phosphoric acid in methanol. In some embodiments, a compound of Formula VII is treated with phosphoric acid in acetone. In some embodiments, a compound of Formula VII is treated with phosphoric acid in water. In some embodiments, a compound of Formula VII is treated with phosphoric acid with no additional solvent. In some embodiments, the phosphoric acid is an 85% aqueous solution of phosphoric acid. In some embodiments, the phosphoric acid is neat phosphoric acid.

In some embodiments, a compound of Formula VII is treated with phosphoric acid at a reaction temperature of about 30° C. to about 45° C. In some embodiments, a compound of Formula VII is treated with phosphoric acid at a reaction temperature of about 35° C. to about 40° C. In some embodiments, a compound of Formula VII is treated with phosphoric acid at a reaction temperature of about 35° C. Lastly, the compound of Formula VIII is isolated by crystallization.

In some embodiments, the compound of Formula VIII is re-crystallized.

In some embodiments, the compound of Formula VIII is crystallized from acetonitrile and water. In some embodiments, the compound of Formula VIII is crystallized from acetonitrile and water in about a 2:3 ratio of acetonitrile to water (% v/v). In some embodiments, the compound of Formula VIII is crystallized from acetonitrile and water at a pH of about 8.0 to about 9.5. In some embodiments, the compound of Formula VIII is crystallized from acetonitrile and water at a pH of about 8.0. In some embodiments, the compound of Formula VIII is crystallized from acetonitrile and water at a pH of about 9.5.

In some embodiments, the compound of Formula VIII is re-crystallized. In some embodiments, the compound of Formula VIII is re-crystallized from trifluoroethanol and ethanol; acetone and water; NMP and water; NMP and toluene; dimethylsulfoxide and toluene or acetonitrile and water. In some embodiments, the compound of Formula VIII is re-crystallized from trifluoroethanol and ethanol in about a 1:2 ratio of trifluoroethanol to ethanol (% v/v).

A number of intermediates have been made that demonstrate improved properties and handling over intermediates previously described. Thus, in another aspect, a compound of the disclosure is a compound having Formula IX:

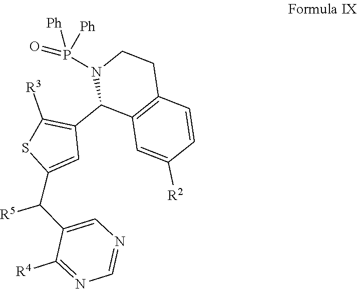

wherein
R² is selected from H and halogen;
R³ is selected from H and alkyl;
R⁴ is selected from H, halogen, and —NR⁶R⁷;
R⁵ is selected from —OH and =O;
R⁶ and R⁷ are independently selected from H, Formula X, and Formula XI:

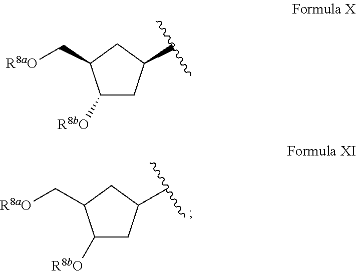

R^{8a} and R^{8b} are independently selected from H, triisopropylsilyl, trimethylsilyl, triethylsilyl, tert-butyldimethylsilyl, tert-butyldiphenylsilyl, and —S(O)₂NR⁹R¹⁰; and
R⁹ and R¹⁰ are independently selected from −1 and alkyl.

In another aspect, a compound of the disclosure is a compound having Formula IX; wherein
R² is selected from H and halogen;
R³ is selected from H and alkyl;
R⁴ is Cl; and
R⁵ is ═O.

In another aspect, a compound of the disclosure is a compound having Formula IX; wherein
R² is selected from H and halogen;
R³ is selected from H and alkyl;
R⁴ is —NR⁶R⁷;
R⁵ is ═O;
R⁶ and R⁷ are independently selected from H, Formula X, and Formula XI:

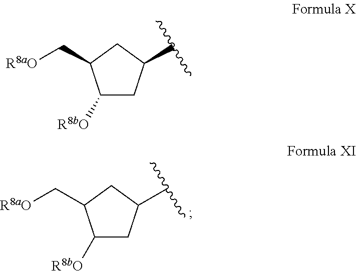

R^{8a} and R^{8b} are independently selected from H, triisopropylsilyl, trimethylsilyl, triethylsilyl, tert-butyldimethylsilyl, tert-butyldiphenylsilyl, and —S(O)₂NR⁹R¹⁰; and
R⁹ and R¹⁰ are independently selected from H and alkyl.

In some embodiments, a compound of the disclosure is a compound having Formula IX; wherein
R² is selected from H and halogen;
R³ is selected from H and alkyl;
R⁴ is —NR⁶R⁷;
R⁵ is ═O;
R⁶ is H;
R⁷ is Formula X:

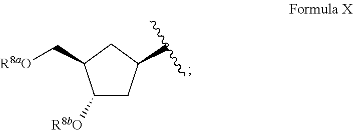

R^{8a} is —S(O)₂NR⁹R¹⁰;
R^{8b} is triisopropylsilyl; and
R⁹ and R¹⁰ are H.

In some embodiments, a compound of the disclosure is a compound having Formula XII:

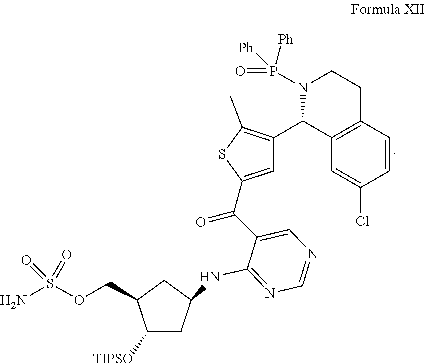

In some embodiments, a compound of the disclosure is a compound having Formula XIII:

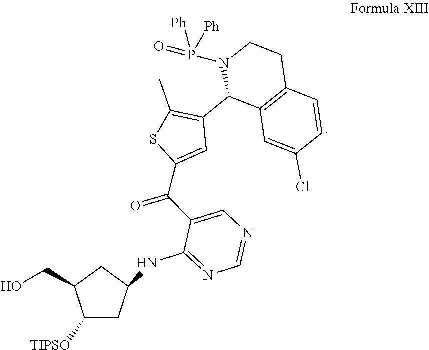

In some embodiments, a compound of the disclosure is a compound having Formula XIV:

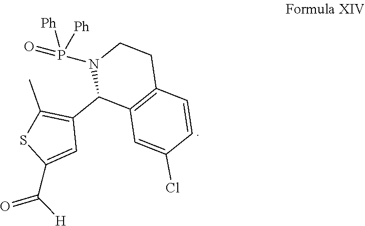 [SMILES: Cc1sc(C=O)cc1[C@H]1c2cc(Cl)ccc2CCN1P(=O)(c1ccccc1)c1ccccc1]

In some embodiments, a compound of the disclosure is a compound having Formula XV:

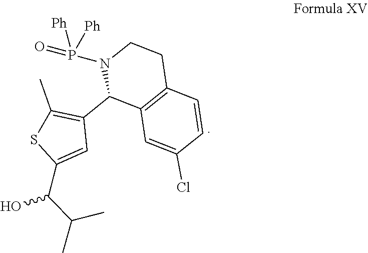 [SMILES: Cc1sc(C(O)C(C)C)cc1[C@H]1c2cc(Cl)ccc2CCN1P(=O)(c1ccccc1)c1ccccc1]

In some embodiments, a compound of the disclosure is a compound having Formula XVI:

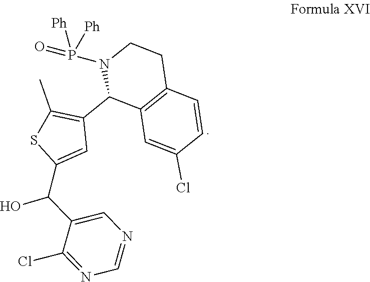 [SMILES: Cc1sc(C(O)c2cncnc2Cl)cc1[C@H]1c2cc(Cl)ccc2CCN1P(=O)(c1ccccc1)c1ccccc1]

In some embodiments, a compound of the disclosure is a compound having Formula XVII:

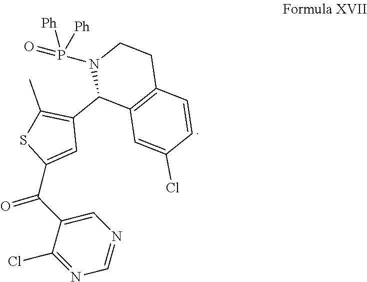 [SMILES: Cc1sc(C(=O)c2cncnc2Cl)cc1[C@H]1c2cc(Cl)ccc2CCN1P(=O)(c1ccccc1)c1ccccc1]

### Definitions

As used herein, the singular form “a,” “an,” and “the” includes plural references unless indicated otherwise. Use of the term “or” herein is not meant to imply that alternatives are mutually exclusive.

In this application, the use of “or” means “and/or” unless expressly stated or understood by one skilled in the art. In the context of a multiple dependent claim, the use of “or” refers back to more than one preceding independent or dependent claim.

The term “about,” as used herein, includes the recited number ±10%. Thus, “about 10” means 9 to 11. As is understood by one skilled in the art, reference to “about” a value or parameter herein includes (and describes) instances that are directed to that value or parameter per se. For example, description referring to “about X” includes description of “X.”

For the purpose of the present disclosure, the term “halo” or “halogen” as used by itself or as part of another group refers to a halogen atom. Non-limiting exemplary halo groups include fluoro, chloro, bromo, and iodo.

For the purpose of the present disclosure, the term “alkyl” as used by itself or as part of another group refers to a straight- or branched-chain aliphatic hydrocarbon containing one to twelve carbon atoms (i.e., C₁₋₁₂ alkyl) or the number of carbon atoms designated (i.e., a C₁ alkyl such as methyl, a C₂ alkyl such as ethyl, a C₃ alkyl such as propyl or isopropyl, etc.). The alkyl group can be suitably chosen from a straight chain C₁₋₁₀ alkyl group, a branched chain C₃₋₁₀ alkyl group, a straight chain C₁₋₆ alkyl group, a branched chain C₃₋₆ alkyl group, a straight chain C₁₋₄ alkyl group, a branched chain C₃₋₄ alkyl group, a straight or branched chain C₃₋₄ alkyl group. The alkyl group can be partially or completely deuterated, i.e., one or more hydrogen atoms of the alkyl group are replaced with deuterium atoms. Non-limiting exemplary alkyl groups include methyl (including —CD₃), ethyl, propyl, isopropyl, butyl, sec-butyl, tert-butyl, iso-butyl, 3-pentyl, hexyl, heptyl, octyl, nonyl, and decyl.

For the purpose fo the present disclosure, the term “continous flow” or “flow chemistry” refers to a continuous processing, which begins with two or more streams of different reactants pumped at specific flow rates into a single chamber, tube, or microreactor. A reaction takes place, and the stream containing the resultant compound is collected at the outlet.

For the purpose of the present disclosure, the term “% AUC” as used herein means percent area under the curve. The % AUC refers to a method for determining relative amounts of particular products following a chemical reaction as shown by the area under a curve resulting from high performance liquid chromatography (HPLC) purification and/or measurements. For example, a reaction that results in a compound of Formula I in an amount of less than or equal to 3% AUC as compared to a compound of Formula III, means that a compound of Formula I is formed in an amount that is less than or equal to 3% of the amount of a compound Formula II that is formed, as shown by the area under the curve following normalized integration of the peaks corresponding to Formula I and Formula III.

For the purpose of the present disclosure, the term “polar aprotic organic solvent” as used herein means a solvent that lacks an acidic proton and is polar. Non-limiting exemplary polar aprotic organic solvents include THF, 2-MeTHF, CPME, dimethylformamide, DCM, dimethylsulfoxide, ethyl acetate, acetonitrile, acetone, and diethyl ether.

### EXAMPLES

### Example 1: ((1R)-7-chloro-1-(5-((4-chloropyrimidin-5-yl)(hydroxy)methyl)-2-methylthiophen-3-yl)-3,4-dihydroisoquinolin-2(1H)-yl)diphenylphosphine oxide (Formula XVI)

Solutions of (R)-4-(7-chloro-2-(diphenylphosphoryl)-1,2,3,4-tetrahydroisoquinolin-1-yl)-5-methylthiophene-2-carbaldehyde (20 g, 1.00 eq. LR) in 130 mL of THF and 4-chloro-5-iodopyrimidine (15.6 g, 1.60 eq.) in 160 mL of THF were prepared by weighing solids into separate pre-dried reaction vessel, charging with the required amount of anhydrous THE and sonicating to obtain clear solutions. After sonication, the solutions were filtered to remove any insoluble matter. The water content of the two solutions were assessed by Karl-Fisher Titration (target water content: <500 ppm (0.05%)). A solution of iPrMgCl·LiCl (46.9 mL of 1.3 M solution in THF; 1.50 eq.) was added to a predried vessels under a blanket of nitrogen. The reactor system was dried by flushing with anhydrous THE until the KF of solvent leaving the reactor was <500 ppm. The reactor and reagent feed streams were cooled to 0° C. The reagent feed lines were primed with the respective reagent streams and then 4-chloro-5-iodopyrimidine, iPrMgCl·LiCl, and (R)-4-(7-chloro-2-(diphenylphosphoryl)-1,2,3,4-tetrahydroisoquinolin-1-yl)-5-methylthiophene-2-carbaldehyde solution flow was started at the desired rates. After the feed streams for 4-chloro-5-iodopyrimidine and iPrMgCl·LiCl are combined the Isopropyl magnesium chloride-lithium chloride complex in THF was titrated to determine its potency. The resultant reaction stream was diverted to waste for a minimum of 5 residence times (with respect to the Coupling step) to allow the system to reach steady-state. After 5 residence times, a sample was removed and a reaction in process control sample analysis (IPC) taken. The pumps were stopped during the data collection of the reaction IPC. After a successful reaction IPC, the pumps were started and again the output stream was diverted to waste for 5 residence times. After 5 residence times, the reaction outflow was collected in the quench continuous stirred tank reactor (CSTR) along with a solution of 10% w/w aq. Ammonium chloride solution to continuously quench the reaction while maintaining the temperature at a target of 5° C.

After completion of the reaction, the phases were separated, and the aqueous phase was rejected. The organic phase was washed with 25% w/w aq. sodium chloride solution and the layers were separated and the aqueous layer was rejected. The organic layer was filtered through a pad of Celite, followed by a THE rinse of the CELITE pad, before being assayed by HPLC for ((1R)-7-chloro-1-(5-((4-chloropyrimidin-5-yl)(hydroxy)methyl)-2-methylthiophen-3-yl)-3,4-dihydroisoquinolin-2(1H)-yl)diphenylphosphine oxide content. The filtrates were distilled down to 6.5 volumes and Ethanol (15 mL/g) was added. The mixture was further distilled to a target of 10 mL/g. A THE content IPC is taken (<5.0 wt %). The mixture was cooled down to 20° C. and 1 wt % of seed was added and held for 1 hour. To the reactor, 10 mL/g of water was added over 6 hours and the resulting slurry was aged for 6 hours before the product is isolated by filtration. The filter cake was washed once with of (1:1) ethanol/water (3 mL/g) and three times with n-heptane (3 mL/g) before being dried under vacuum at 40° C. to give ((1R)-7-chloro-1-(5-((4-chloropyrimidin-5-yl)(hydroxy)methyl)-2-methylthiophen-3-yl)-3,4-dihydroisoquinolin-2(1H)-yl)diphenylphosphine oxide as a mixture of two diastereomers, with a ratio of 1:0.27, and two rotational isomers, with a ratio of 1:1. Major Isomer: ¹H-NMR (400 MHz, DMSO-d₆) δ 9.02 (s, 1H), 8.90 (s, 1H), 7.62-7.36 (m, 9H), 7.27-7.21 (m, 3H), 6.92 (d, J=1.9 Hz, 1H), 6.64 (s, 1H), 6.61 (d, J=4.2 Hz, 1H), 6.00 (d, J=4.2 Hz, 1H), 5.76 (d, J=7.7 Hz, 1H), 3.47-3.34 (m, 1H), 3.20-3.10 (m, 1H), 2.71-2.60 (m, 2H), 2.08 (s, 3H). ¹³C-NMR (100 MHz, DMSO-d₆) δ 158.5, 158.1, 156.7, 142.0, 139.2, 138.4, 136.4, 133.5, 133.3, 132.9, 132.6, 132.2, 132.1, 132.0, 131.9, 131.7, 131.6, 130.8, 129.3, 128.7, 127.6, 127.1, 126.9, 66.3, 49.9, 38.7, 27.9, 13.4. HRESIMS: m/z 606.0923 [M+H](calculated for C₃₁H₂₇C₂N₃O₂PS⁺, 606.0933).

### Example 2: Synthesis of (R)-(4-(7-chloro-2-(diphenylphosphoryl)-1,2,3,4-tetrahydroisoquinolin-1-yl)-5-methylthiophen-2-yl)(4-chloropyrimidin-5-yl)methanone (Formula XVII)

((1R)-7-Chloro-1-(5-((4-chloropyrimidin-5-yl)(hydroxy)methyl)-2-methylthiophen-3-yl)-3,4-dihydroisoquinolin-2(1H)-yl)diphenylphosphine oxide (100 g, 1 eq.) was added to the reaction vessel along with THE (300 mL) at ambient temperature. TEMPO (2.47 g, 0.1 eq), NaHCO₃ (14.3 g, 1.1 eq) and potassium bromide (3.68 g, 0.2 eq) were added to the reaction vessel. The reaction vessel was cooled to <5° C. A 15.75 wt % solution of sodium hypochlorite in water (2.0 eq) was added slowly over 1 hour. The color changed from dark orange to light orange upon addition of the sodium hypochlorite. The reaction temperature was maintained <5° C. for 1-3 hour. Upon reaching the completion, reaction was quenched with 5 volumes (500 mL) of 5 wt % aqueous Na₂SO₃. The layers were separated and the organic layer was washed with 5 volumes (500 mL) of 25% NaCl. Organic layer was separated and concentrated to around 3 volumes (300 mL). The resulting product (R)-(4-(7-chloro-2-(diphenylphosphoryl)-1,2,3,4-tetrahydroisoquinolin-1-yl)-5-methylthiophen-2-yl)(4-chloropyrimidin-5-yl)methanone was isolated as THF solution and used in the next step (Example 3).

### Example 3: Synthesis of (4-((R)-7-chloro-2-(diphenylphosphoryl)-1,2,3,4-tetrahydroisoquinolin-1-yl)-5-methylthiophen-2-yl)(4-(((1R,3R,4S)-3-(hydroxymethyl)-4-((triisopropylsilyl)oxy)cyclopentyl)amino)pyrimidin-5-yl)methanone (Formula XIII)

Potassium carbonate (6.0 eq) was added to the reaction vessel with 300 mL of water and dissolved at ambient temperature. ((1R,2S,4R)-4-Amino-2-((triisopropylsilyl)oxy)cyclopentyl)methanol H₃PO₄ (1.05 eq) in 200 mL of THE was added. The mixture was stirred at ambient temperature until all solid dissolved. The THF solution (R)-(4-(7-chloro-2-(diphenylphosphoryl)-1,2,3,4-tetrahydroisoquinolin-1-yl)-5-methylthiophen-2-yl)(4-chloropyrimidin-5-yl)methanone (1.0 eq.) from Example 2 was added. The reaction was stirred and heated to 45° C. Upon reaching completion, the layers were separated and the organic layer was subjected to put/take azeotropic distillation until Karl Fisher analysis was below 0.4%. The resulting (4-((R)-7-chloro-2-(diphenylphosphoryl)-1,2,3,4-tetrahydroisoquinolin-1-yl)-5-methylthiophen-2-yl)(4-(((1R,3R,4S)-3-(hydroxymethyl)-4-((triisopropylsilyl)oxy)cyclopentyl)amino)pyrimidin-5-yl)methanone crude solution was used in the next step (Example 4).

### Example 4: Synthesis of ((1R,2S,4R)-4-((5-(4-((R)-7-chloro-2-(diphenylphosphoryl)-1,2,3,4-tetrahydroisoquinolin-1-yl)-5-methylthiophene-2-carbonyl)pyrimidin-4-yl)amino)-2-((triisopropylsilyl)oxy)cyclopentyl)methyl sulfamate (Formula XII)

Sulfamoyl chloride (3.00 eq.) is added to the reaction vessel with N-methyl-2-pyrrolidinone (2.5 L/kg). The slurry is cooled to 0° C. Separately, (4-((R)-7-chloro-2-(diphenylphosphoryl)-1,2,3,4-tetrahydroisoquinolin-1-yl)-5-methylthiophen-2-yl)(4-(((1R,3R,4S)-3-(hydroxymethyl)-4-((triisopropylsilyl)oxy)cyclopentyl)amino)pyrimidin-5-yl)methanone (1 eq. LR) as a solution in THF is charged to N-methyl-2-pyrrolidinone (2.5 L/kg) and distilled to remove THF. Pyridine (3.0 eq.) is added to the (4-((R)-7-chloro-2-(diphenylphosphoryl)-1,2,3,4-tetrahydroisoquinolin-1-yl)-5-methylthiophen-2-yl)(4-(((1R,3R,4S)-3-(hydroxymethyl)-4-((triisopropylsilyl)oxy)cyclopentyl)amino)pyrimidin-5-yl)methanone solution in N-methyl-2-pyrrolidinone. The (4-((R)-7-chloro-2-(diphenylphosphoryl)-1,2,3,4-tetrahydroisoquinolin-1-yl)-5-methylthiophen-2-yl)(4-(((1R,3R,4S)-3-(hydroxymethyl)-4-((triisopropylsilyl)oxy)cyclopentyl)amino)pyrimidin-5-yl)methanone solution is added to the sulfamoyl chloride slurry over 1 h. The reaction mixture is stirred at 0° C. for 0.3-3.0 hours. After >99% conversion to ((1R,2S,4R)-4-((5-(4-((R)-7-chloro-2-(diphenylphosphoryl)-1,2,3,4-tetrahydroisoquinolin-1-yl)-5-methylthiophene-2-carbonyl)pyrimidin-4-yl)amino)-2-((triisopropylsilyl)oxy)cyclopentyl)methyl sulfamate is confirmed, isopropyl acetate (5.0 L/kg, 4.4 kg/kg) and saturated aqueous sodium bicarbonate (5.0 L/kg) are added to the reaction mixture. The reaction mixture is stirred and warmed to 25° C. and the layers are separated. Saturated aqueous sodium bicarbonate (5.0 L/kg) is added again to the organic layer. The biphasic mixture is stirred and the layers are separated. Saturated aqueous sodium chloride (2.0 L/kg) and water (3.0 L/kg) are added to the organic layer. The biphasic mixture is stirred and the layers are separated. The organic layer is distilled to a volume of 3 L/kg. A plug of silica gel (3.0 kg/kg) is packed with ethyl acetate. The concentrated solution is loaded onto the silica plug and eluted with ethyl acetate, collecting the eluent in fractions. The fractions containing product of acceptable purity (>92.0%) are combined and distilled to a volume of 5 L/kg. The ethyl acetate solution of product is dosed into heptane (25 L/kg) at 5° C. over 2 h. The product is isolated by filtration, washed twice with heptane (3 L/kg each), and dried under vacuum at 40° C. to obtain ((1R,2S,4R)-4-((5-(4-((R)-7-chloro-2-(diphenylphosphoryl)-1,2,3,4-tetrahydroisoquinolin-1-yl)-5-methylthiophene-2-carbonyl)pyrimidin-4-yl)amino)-2-((triisopropylsilyl)oxy)cyclopentyl)methyl sulfamate as a mixture of two rotational isomers, with a ratio of 1:1. ¹H-NMR (400 MHz, DMSO-d₆) δ 8.58 (s, 1H), 8.42 (s, 1H), 8.18 (d, J=7.5 Hz, 1H), 7.70-7.41 (m, 10H), 7.24-7.22 (m, 2H), 7.13 (s, 1H), 6.99 (d, J=1.9 Hz, 1H), 5.87 (d, J=7.7 Hz, 1H), 4.73 (h, J=8.2 Hz, 1H), 4.23 (dt, J=6.8, 3.5 Hz, 1H), 4.05 (dd, J=9.9, 6.5 Hz, 1H), 3.98 (dd, J=9.9, 6.6 Hz, 1H), 3.37-3.28 (m, 1H), 3.20-3.10 (m, 1H), 2.72-2.66 (m, 2H), 2.35 (dt, J=12.7, 7.8 Hz, 1H), 2.23 (s, 3H), 2.18 (tt, J=6.4, 3.7 Hz, 1H), 2.00-1.95 (m, 1H), 1.82 (ddd, J=12.8, 9.1, 5.9 Hz, 1H), 1.31-1.24 (m, 1H), 1.03 (s, 18H), 1.02 (s, 3H). ¹³C-NMR (100 MHz, DMSO-d₆) δ 185.8, 160.8, 159.8, 158.3, 147.2, 140.9, 138.4, 138.2, 136.7, 133.6, 132.9, 132.7, 132.3, 132.2, 132.1, 131.7, 131.6, 131.0, 129.4, 129.1, 127.6, 127.4, 112.4, 73.9, 70.5, 50.6, 49.2, 47.2, 41.4, 38.8, 33.7, 28.8, 18.3, 14.4, 12.1. HRESIMS: m/z 934.3008 [M+H] (calculated for C₄₆H₅₈ClN₅O₆PSSi⁺, 934.3018).

### Example 5: Synthesis of ((1R,2S,4R)-4-((5-(4-((R)-7-chloro-1,2,3,4-tetrahydroisoquinolin-1-yl)-5-methylthiophene-2-carbonyl)pyrimidin-4-yl)amino)-2-hydroxycyclopentyl)methyl sulfamate (Formula VIII)

((1R,2S,4R)-4-((5-(4-((R)-7-Chloro-2-(diphenylphosphoryl)-1,2,3,4-tetrahydroisoquinolin-1-yl)-5-methylthiophene-2-carbonyl)pyrimidin-4-yl)amino)-2-((triisopropylsilyl)oxy)cyclopentyl)methyl sulfamate (lg, 1.0 eq.) is placed in a reaction vessel. Acetonitrile (2 mL) followed by phosphoric acid (85 wt % in water, 2 mL, 27 equiv) is is added to the reaction vessel. The resulting mixture is stirred at 35° C. for 24 hours. IPC analysis to establish target conversion of <0.50% ((1R,2S,4R)-4-((5-(4-((R)-7-chloro-2-(diphenylphosphoryl)-1,2,3,4-tetrahydroisoquinolin-1-yl)-5-methylthiophene-2-carbonyl)pyrimidin-4-yl)amino)-2-((triisopropylsilyl)oxy)cyclopentyl)methyl sulfamate or mono (TIPS) deprotected intermediate remaining. The mixture is then cooled to 20° C. Acetonitrile (4 mL) is added to the reaction vessel and the reaction mixture is stirred. Sodium carbonate (20 wt % in water, 8 mL), is added to the reaction mixture via dosing pump over 1 hour, controlling off-gassing as necessary. After the quench is complete, the agitation is stopped, and the biphasic mixture is allowed to separate (both layers are approx. pH 5). The aqueous phase (bottom layer) is separated and rejected. Water (9 mL) is mixed with ammonium hydroxide (28 mass % in water) (0.3 mL) in a separate vessel. The mixture of water:ammonium hydroxide (4 mL) is added to the agitated organic layer to adjust the pH of the mixture to approximately pH 8. The resulting solution is seeded with ((1R,2S,4R)-4-((5-(4-((R)-7-chloro-1,2,3,4-tetrahydroisoquinolin-1-yl)-5-methylthiophene-2-carbonyl)pyrimidin-4-yl)amino)-2-hydroxycyclopentyl)methyl sulfamate anhydrate form (1 wt %) and agitated for 3 hours. To the resulting slurry, the rest of water:ammonium hydroxide mixture (5.3 mL) is added over 2.5 hours via dosing pump and the mixture is agitated for >18 hours. The solids are filtered, washed with 2×2 volumes 2:3/MeCN:Water. The filtered solids are dried in a vacuum oven overnight at 45° C. to give a typical yield of >90% of (((1R,2S,4R)-4-((5-(4-((R)-7-chloro-1,2,3,4-tetrahydroisoquinolin-1-yl)-5-methylthiophene-2-carbonyl)pyrimidin-4-yl)amino)-2-hydroxycyclopentyl)methyl sulfamate) as a light yellow solid.

### Example 6: Recrystallization of ((1R,2S,4R)-4-((5-(4-((R)-7-chloro-1,2,3,4-tetrahydroisoquinolin-1-yl)-5-methylthiophene-2-carbonyl)pyrimidin-4-yl)amino)-2-hydroxycyclopentyl)methyl sulfamate (Formula VIII)

((1R,2S,4R)-4-((5-(4-((R)-7-Chloro-1,2,3,4-tetrahydroisoquinolin-1-yl)-5-methylthiophene-2-carbonyl)pyrimidin-4-yl)amino)-2-hydroxycyclopentyl)methyl sulfamate (freebase, 3 g, 1 equiv) and trifluoroethanol (TFE, 10 L/kg, 7.6 kg/kg) were added to a clean, dry, and inert reaction vessel. The mixture was agitated and heated to 35° C. until dissolution. The solution was filtered and the resulting solution is added to a new clean, dry, and inert reaction vessel. The solution is cooled to 20° C. Ethanol (EtOH, 19.5 L/kg, 15.4 kg/kg) is added to a separate clean, dry, and inert reaction vessle. The vessel is heated to 25° C. and agitated. ((1R,2S,4R)-4-((5-(4-((R)-7-chloro-1,2,3,4-tetrahydroisoquinolin-1-yl)-5-methylthiophene-2-carbonyl)pyrimidin-4-yl)amino)-2-hydroxycyclopentyl)methyl sulfamate seeds (0.05 kg/kg) suspended in EtOH (0.5 L/kg, 0.4 kg/kg) are added to vessel. The clarified ((1R,2S,4R)-4-((5-(4-((R)-7-chloro-1,2,3,4-tetrahydroisoquinolin-1-yl)-5-methylthiophene-2-carbonyl)pyrimidin-4-yl)amino)-2-hydroxycyclopentyl)methyl sulfamatein TFE solution is added to the EtOH containing vessel over 1 hour. The slurry is held at 25° C. for 24 hours, until a specification of ((1R,2S,4R)-4-((5-(4-((R)-7-chloro-1,2,3,4-tetrahydroisoquinolin-1-yl)-5-methylthiophene-2-carbonyl)pyrimidin-4-yl)amino)-2-hydroxycyclopentyl)methyl sulfamate <5 mg/mL is achieved. The solids are collected by filtration, washed with 1:2 TFE/EtOH v/v % (5 L/Kg), followed by an 1×EtOH washes (5×L/Kg) and dried under vacuum at 50° C. to give re-crystallized ((1R,2S,4R)-4-((5-(4-((R)-7-chloro-1,2,3,4-tetrahydroisoquinolin-1-yl)-5-methylthiophene-2-carbonyl)pyrimidin-4-yl)amino)-2-hydroxycyclopentyl)methyl sulfamate (2.7 g, 0.9 eq.). The analytical data (NMR and MS) for ((1R,2S,4R)-4-((5-(4-((R)-7-chloro-1,2,3,4-tetrahydroisoquinolin-1-yl)-5-methylthiophene-2-carbonyl)pyrimidin-4-yl)amino)-2-hydroxycyclopentyl)methyl sulfamate matches the data that was previously disclosed in published International Appl. No. WO 2016/004136 for this compound.

It is to be understood that the foregoing described embodiments and exemplifications are not intended to be limiting in any respect to the scope of the disclosure, and that the claims presented herein are intended to encompass all embodiments and exemplifications whether or not explicitly presented herein

All patents and publications cited herein are fully incorporated by reference in their entirety.

What is claimed is:

1. A process, comprising:
reacting a compound of Formula I: 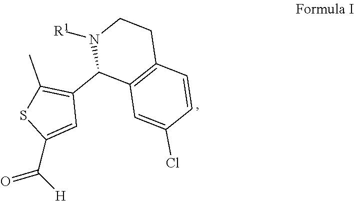
wherein R¹ is selected from diphenylphosphine oxide (DPP), dibenzyl suberyl (DBS), trifluoroacetate (TFA) or —C(O)O^{t}Bu;
with a compound of Formula II 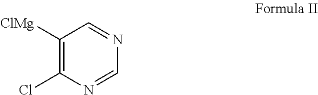
in a solvent while maintaining a reaction temperature of about −20° C. to about 10° C. to form the compound of Formula III: 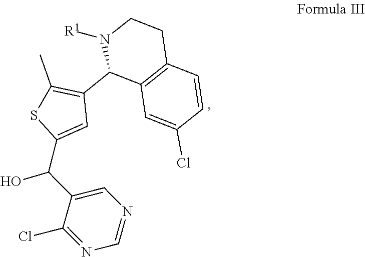
wherein R¹ is as defined above; and
wherein the compound of Formula II is prepared by reacting 4-chloro-5-iodopyrimidine with isopropyl magnesium chloride-lithium chloride complex (i-PrMgCl·LiCl) in an anhydrous solvent while maintaining a reaction temperature of about 0° C. to about 5° C.;
adding an acidic aqueous solution to quench the reaction; and
isolating the compound of Formula III.

2. The process of claim 1, wherein R¹ is DPP.

3. The process of claim 1, wherein the solvent is one or more polar aprotic organic solvents selected from tetrahydrofuran (THF), 2-methyl-tetrahydrofuran (2-MeTHF), cyclopentyl methyl ether (CPME), and diethyl ether.

4. The process of claim 3, wherein the one or more solvents is THF.

5. The process of claim 4, wherein 4-chloro-5-iodopyrimidine in THE is combined with i-PrMgCl·LiCl in TIHIF; and the mixture of 4-chloro-5-iodopyrimidine and i-PrMgCl·LiCl in THF is thereafter combined with the compound of Formula I in THF to form the compound of Formula III.

6. The process of claim 1, wherein the reaction is quenched using an aqueous acid solution.

7. The process of claim 1, wherein the reaction temperature is maintained from about 0° C. to about 5° C.

8. The process of claim 1, wherein the compound of Formula III is isolated by crystallization.

9. The process of claim 8, wherein the compound of Formula III is crystallized from ethanol and water.

10. The process of claim 1, further comprising the process of preparing the compound of Formula VIII, comprising;
oxidizing the compound of Formula III in one or more solvents to form the compound of Formula IV; 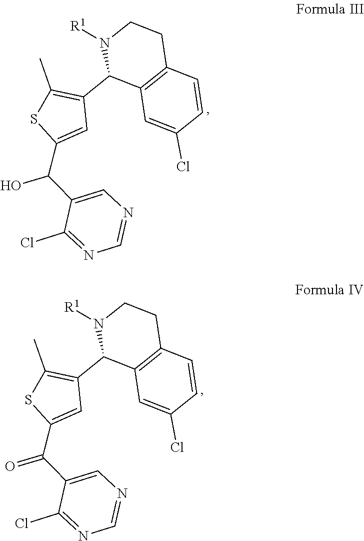
reacting the compound of Formula IV with the compound of Formula V in one or more solvents to form the compound of Formula VI: 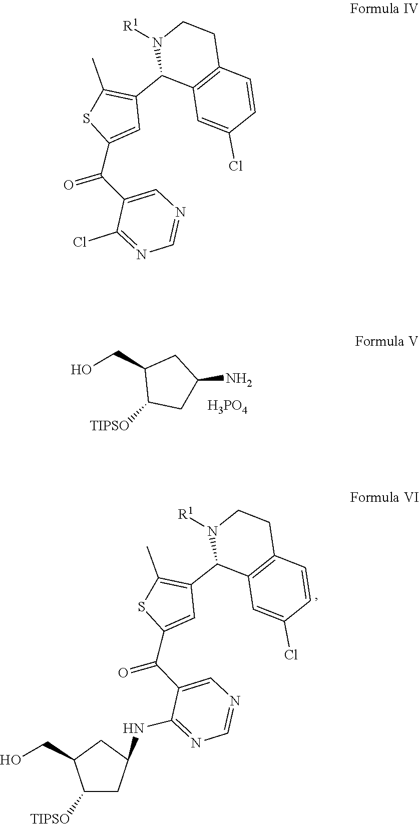
reacting the compound of Formula VI with sulfamoyl chloride in one or more solvents to form the compound of Formula VII: 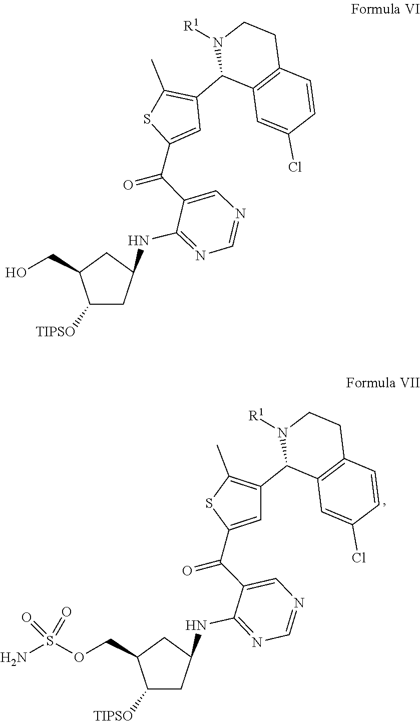
treating the compound of Formula VII with phosphoric acid in one or more solvents to form the compound of Formula VIII; 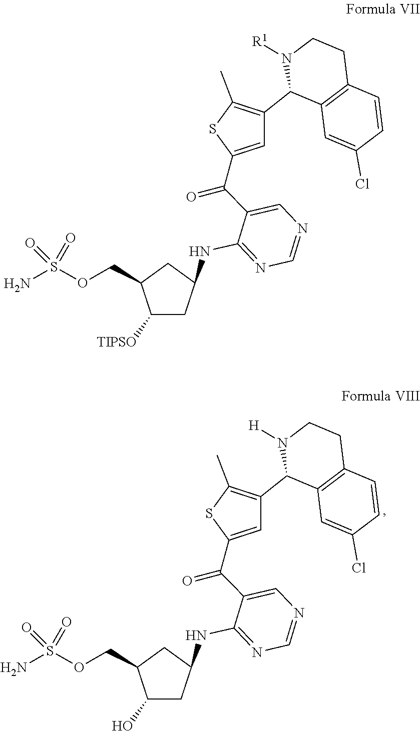
and
isolating the compound of Formula VIII by crystallization;
wherein for each of Formula III, IV, V, VI and VII R¹ is DPP, DBS, TFA or —C(O)O^{t}Bu.

11. The process of claim 10, further comprising re-crystallizing the compound of Formula VIII.

12. The process of claim 10, wherein the compound of Formula III is oxidized using hypochlorite and a catalyst selected from 9-Azabicyclo[3.3.1]nonane N-Oxyl (ABNO), 2-Azaadamantane N-Oxyl (AZADO), 4-hydroxy-2,2,6,6-tetramethylpiperidin-1-oxyl (hydroxyl-TEMPO) and 2,2,6,6-tetramethylpiperidinyloxy (TEMPO).

13. The process of claim 10, wherein the compound of Formula III is oxidized in a solvent selected from THF, 2-MeTHF, dichloromethane (DCM), toluene, methyl ethyl ketone (MEK), methyl iso-butyl ketone (MIBK), acetone, 1-methyl-2-pyrrolidinone (NMP), N,N-dimethylformamide (DMF), and combinations thereof.

14. The process of claim 10, wherein the compound of Formula IV is reacted with the compound of Formula V in a solvent selected from THF, 2-MeTHF, DCM, methanol (MeOH), water and combinations thereof.

15. The process of claim 10, wherein the compound of Formula VI is reacted with sulfamoyl chloride in a solvent selected from N-methyl-2-pyrrolidone, pyridine, 2-MeTHF, THF, DMF, N,N-dimethylacetamide (DMAc), water and combinations thereof.

16. The process of claim 10, wherein the compound of Formula VII is treated with phosphoric acid in a solvent selected from acetonitrile, isopropanol, ethanol, methanol, acetone, water and combinations thereof.

17. The process of claim 4, wherein the process is conducted in a continuous flow reactor.

18. The process of claim 10, wherein the steps to prepare the compound of Formula III are conducted in a continuous flow reactor.

19. A compound of Formula IX: 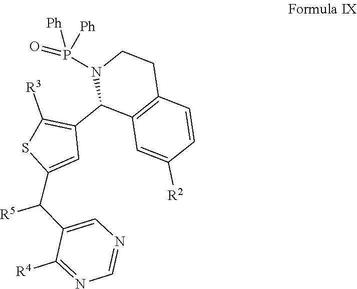
wherein
R² is selected from H and halogen;
R³ is selected from H and alkyl;
R⁴ is selected from H, halogen, and —NR⁶R⁷;
R⁵ is selected from —OH and =O;
R⁶ and R⁷ are independently selected from H, Formula X, and Formula XI: 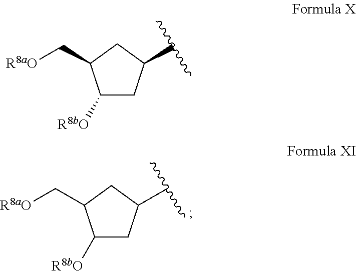
R^{8a} and R^{8b} are independently selected from H, triisopropylsilyl, trimethylsilyl, triethylsilyl, tert-butyldimethylsilyl, tert-butyldiphenylsilyl, and —S(O)₂NR⁹R¹⁰; and
R⁹ and R¹⁰ are independently selected from H and alkyl.

20. The compound of claim 19, wherein:
R² is selected from H and halogen;
R³ is selected from H and alkyl;
R⁴ is Cl; and
R⁵ is ═O.

21. The compound of claim 19, wherein:
R² is selected from H and halogen;
R³ is selected from H and alkyl;
R⁴ is —NR⁶R⁷;
R⁵ is =O;
R⁶ and R⁷ are independently selected from H, Formula X, and Formula XI: 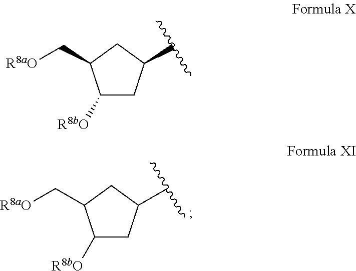
R^{8a} and R^{8b} are independently selected from H, triisopropylsilyl, trimethylsilyl, triethylsilyl, tert-butyldimethylsilyl, tert-butyldiphenylsilyl, and —S(O)₂NR⁹R¹⁰; and
R⁹ and R¹⁰ are independently selected from H and alkyl.

22. The compound of claim 19, wherein:
R² is selected from H and halogen;
R³ is selected from H and alkyl;
R⁴ is —NR⁶R⁷;
R⁵ is =O;
R⁶ is H;
R⁷ is Formula X: 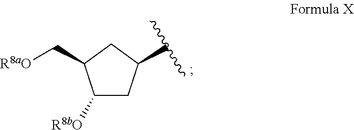
R^{8a} is —S(O)₂NR⁹R¹⁰;
R^{8b} is TIPS; and
R⁹ and R¹⁰ are H.

23. The compound of claim 19, wherein the compound is Formula XII:

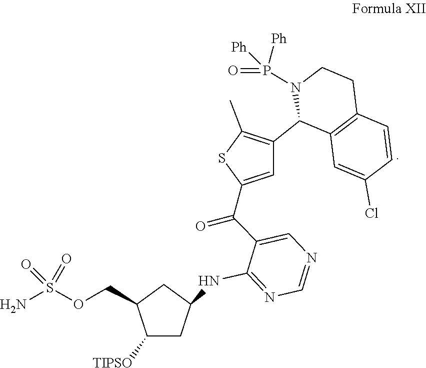

24. The compound of claim 19, wherein the compound is Formula XIII:

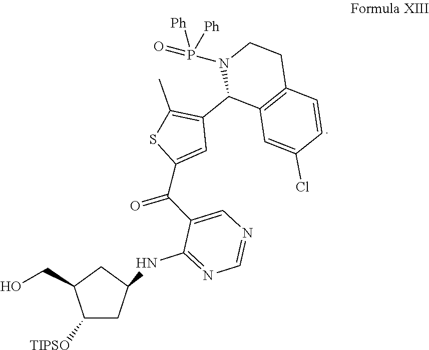

25. A compound of Formula XIV: 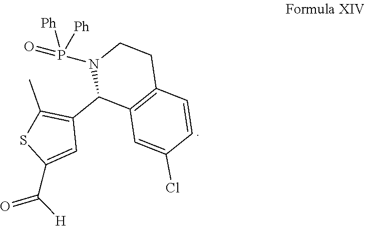 [SMILES: Cc1sc(C=O)cc1[C@H]1c2cc(Cl)ccc2CCN1P(=O)(c1ccccc1)c1ccccc1]
